(19) United States
(12) Patent Application Publication
(10) Pub. No.: US 2016/0220175 A1
(43) Pub. Date: Aug. 4, 2016

(54) APPARATUS AND METHOD FOR RANGE OF MOTION TRACKING WITH INTEGRATED REPORTING

(71) Applicant: The Board of Trustees of The Leland Stanford Junior University, Palo Alto, CA (US)

(72) Inventors: Theoderick Tam, San Jose, CA (US); Michael Jordon Feldstein, Foster City, CA (US); Farzad Azimpour, San Francisco, CA (US); Andrew McGibbon, Menlo Park, CA (US)

(21) Appl. No.: 15/013,839

(22) Filed: Feb. 2, 2016

Related U.S. Application Data
(60) Provisional application No. 62/111,466, filed on Feb. 3, 2015.
(60) Provisional application No. 62/167,829, filed on May 28, 2015.

(51) Int. Cl. A61B 5/00; A61B 5/107; G06F 19/00; A61B 5/11

(52) U.S. Cl. CPC A61B 5/486; A61B 5/11; A61B 5/1071; A61B 5/0004; A61B 5/4528; A61B 5/6829; A61B 5/6824; A61B 5/6823; A61B 5/7282; G06F 19/3418

(57) ABSTRACT

A system for providing information to a patient and receiving information from the patient before and/or after a medical or surgical procedure may include a joint motion sensor system, which may include a first sensor device for coupling with the patient above a joint and including a first transmitter for transmitting sensed data from the first sensor device, and a second sensor device for coupling with the patient below the joint and including a second transmitter for transmitting sensed data from the second sensor device. The system may also include a processor to receive sensed data from the first and second sensor devices and process the sensed data to provide joint motion data. Finally, the system may include a third transmitter coupled with the processor for transmitting the joint motion data wirelessly to the patient.

### CROSS-REFERENCE TO RELATED APPLICATIONS

This application claims priority to U.S. Provisional Patent Application Nos. 62/111,466, filed Feb. 3, 2015, and 62/167,829, filed May 25, 2015. The entireties of each of which are herein incorporated by reference.

### TECHNICAL FIELD

This application is directed to medical devices, systems and methods. More specifically, the application is directed to devices, systems and methods for providing patient specific information and feedback after a surgical or medical intervention to enhance outcomes.

### BACKGROUND

Whenever a patient undergoes a surgical, medical or other interventional procedure, there is a pre-intervention period, an intervention, and a recovery period after the intervention. In most if not all cases, any person undergoing a procedure may benefit from pre-intervention and/or post-intervention education, communication, and information, which may very often enhance the outcome of the intervention. Helpful information that may be provided to a patient and/or his or her healthcare providers and staff, for example, may include data gathered from the patient via patient input, sensors attached to the patient, sensors located in the patient's environment and/or the like. Such information may be provided to the patient in the form of feedback, to help the patient optimize his or her post-intervention outcomes. Unfortunately, healthcare providers and staff who provide such information and communication often do not have enough time or adequate resources to provide a desirable level of education and information.

One example of a post-intervention therapy that is often very helpful in enhancing the outcome of an interventional procedure is physical therapy. One of the challenges with physical therapy, however, is that patients are typically limited in the number and duration of visits that they can have. Another challenge is that physical therapy is often physically demanding and sometimes painful, and thus patient compliance is typically quite low. It is also very difficult to track patient compliance between therapy visits. Another challenge is that therapists often use antiquated instruments, like stopwatches and goniometers, to track patient activity, and they have no way of tracking activity when the patient is out of their site.

One particular example of a surgical procedure that requires post-surgical physical therapy is a total knee replacement (TKR) procedure. Complications of TKR are low, and the device lifetimes of TKR knee implants are long. Approximately 900,000 TKR procedures are performed in the United States every year, and the procedural growth rate is expected to grow at approximately 8.5% per year. Although TKR is a very commonly performed procedure and has high success rates, however, many patients are dissatisfied with the results. In fact, one in five TKR patients is dissatisfied with the results, and one in three patients is dissatisfied with the post-surgical rehabilitation experience. Much of this dissatisfaction may be due to unrealistic expectations of the ease and time of post-surgical recovery and a physical therapy experience that is not optimally managed.

Therefore, it would be desirable to have methods and systems for providing enhanced education, information and/or communication to patients before and/or after a surgical or medical procedure. Ideally, such methods and systems would be easy to use and would provide patients with information that may enhance the outcomes of their procedures. The embodiments described in this application are directed to achieving these objectives.

### SUMMARY

The systems, devices and methods described herein provide useful information through data collection from user input as well as a data collected from sensors on the user in both the pre-event and post-event period. The data collected during these periods is carefully chosen to be informative in making decisions that will alter the course of care and treatment for a given user and hence optimize outcomes. Information is gathered, processed, analyzed, and provided back to the patient, healthcare provider(s) and/or other designated person(s), in order to aid in the decision to change a therapy or behavior. Data may be collected through sensors continuously or may be collected only during specified periods, when the sensors are placed on the patient or turned on automatically or turned on through user or other designated individuals direction. Data may be collected from the user through prompts at specified time intervals or in response to user input or activity. By analyzing the abundant, useful data collected both pre-event and post-event and providing the analyzed data to the user and interested parties, user compliance can be improved through coaching and accountability to themselves and others, while also providing the opportunity for pre- and post-event interventions to be tailored to suit the user's progress to optimize the rate and quality of outcomes. This methodology has distinct advantages over intermittent interaction with individuals that provide education, instruction and motivation, as it is continuous and readily available at all times by taking advantage of mobile technologies such as mobile applications and wearable sensors.

Examples of features provided in various embodiments of the systems and methods described herein include feedback of user derived and sensor derived data to the user and other designated parties, such as physical therapists and physicians. Some embodiments may include notifications, optionally with a snooze option, of when to do exercises and alerts to designated parties if exercises are not performed or are not performed correctly. Some embodiments may be configured to determine if correct exercises are performed and if milestones and metrics of progress are being met. Various embodiments involve measurement of any suitable metrics, such as range of motion, sit-to-stand time, 6-minute walk time, time-to-ascend and descend 7 steps, and the like. Some embodiments may be configured to modify a treatment protocol based on an assessment of activity of a patient who is at home or other location, not in the presence or observation of a provider.

In one aspect, a system for providing information to a patient and receiving information from the patient before and/or after a medical or surgical procedure may include a joint motion sensor system, which may include a first sensor device configured to be coupled with the patient above a joint and including a first transmitter for transmitting sensed data from the first sensor device, and a second sensor device configured to be coupled with the patient below the joint and including a second transmitter for transmitting sensed data from the second sensor device. The system may also include a processor configured to receive sensed data from the first and second sensor devices and process the sensed data to provide joint motion data and a third transmitter coupled with the processor for transmitting the joint motion data wirelessly to the patient.

In some embodiments, the medical or surgical procedure may be a total knee replacement surgery, and the first and second sensor devices may be configured to be coupled above and below a knee joint of the patient. In some embodiments, the joint motion data may reflect an angle of flexion of the knee joint and an angle of extension of the knee joint. In some embodiments, the joint motion sensor system may be configured to be coupled with the patient on either side of a knee, an ankle, a hip, a wrist, an elbow or a shoulder. In some embodiments, the first sensor device and the second sensor device each includes a sensor electronics housing and a base for removably coupling with the housing. The base may include a housing attachment surface and an adhesive surface for attaching to the patient's skin. Optionally, the system may also include an app for providing the joint motion to the patient.

In another aspect, a method for providing information to a patient and receiving information from the patient before and/or after a medical or surgical procedure may involve: receiving patient information in a processor before the patient undergoes the medical or surgical procedure; providing information to the patient via an electronic communication device before the patient undergoes the procedure, at least partially in response to the received patient information; receiving sensed data from at least one sensor attached to the patient after the medical or surgical procedure; processing the sensed data to generate the patient feedback data; and transmitting the patient feedback data wirelessly to the electronic communication device for use by the patient.

In some embodiments, receiving the patient information involves receiving a message from the patient describing a state of a health condition of the patient. In some embodiments, providing information to the patient via the electronic communication device involves providing information via a device such as a smart phone, a tablet, a laptop computer or a desktop computer. In some embodiments, providing information to the patient via the electronic communication device may involve providing a pre-procedure physical therapy plan to the patient. These or other embodiments may also further involve providing a post-procedure physical therapy plan to the patient via the electronic communication device. Such an embodiment may also involve adjusting the post-procedure physical therapy plan, based at least in part on the patient feedback data.

In some embodiments, receiving the sensed data may involve receiving joint motion data from at least two joint motion sensor devices attached to the patient near one of the patient's joints. In some embodiments, the medical or surgical procedure may be a total knee replacement surgery, and the sensed data may be joint motion data related to flexion and extension of one of the patient's knees. In some embodiments, receiving the sensed data may involve receiving joint motion data sensed while the patient is exercising, and the patient feedback data may include data describing whether the patient is performing one or more exercises correctly.

Optionally, some embodiments may also involve transmitting the patient feedback data a physician, a nurse and/or a physical therapist. Also optionally, the method may further include receiving patient input in the processor after the medical or surgical procedure. In some embodiments, receiving the patient information and providing the information may involve receiving and sending text messages to and from the patient, respectively. The method may also optionally include providing patient guidance to the patient after the medical or surgical procedure. Such patient guidance may include, but is not limited to, a post-surgical physical therapy plan, a post-surgical exercise plan, instructional information on how to perform one or more exercises, and an avatar illustrating how the patient is performing one or more exercises. In some embodiments, the method may further involve providing an electronic dashboard to a physical therapist working with the patient to track progress of the patient in a post-surgical physical therapy plan.

These and other aspects and embodiments are described in further detail below, in reference to the attached drawing figures.

### BRIEF DESCRIPTION OF THE DRAWINGS

FIG. 1 is a side view of a knee with a goniometer positioned over it;

FIG. 2 is a side view of a patient's lower limb with two sensor devices positioned around the knee joint, according to one embodiment;

FIG. 3 illustrates a system for communicating with and monitoring a patient, according to one embodiment;

FIG. 4 illustrates a method for engaging with, tracking and enhancing therapy for a patient, according to one embodiment;

FIG. 5 is a front view of a smart phone, showing a provider contacting a patient, according to one embodiment;

FIGS. 6-9 are front views of the smart phone, showing a provider communicating with a patient via text messaging, according to one embodiment;

FIG. 10 is a front view of the smart phone, illustrating a patient education page, according to one embodiment;

FIG. 11 is a front view of the smart phone, illustrating a patient data input page, according to one embodiment;

FIG. 12 illustrates sensor devices for tracking movement of a patient, according to one embodiment;

FIG. 13 is a front view of the smart phone, illustrating a dashboard of tracking information for a patient, according to one embodiment;

FIG. 14 is a front view of the smart phone, illustrating a pre-operative conditioning plan provided to a patient, according to one embodiment;

FIG. 15 is a front view of the smart phone, illustrating a message reminder to a patient, according to one embodiment;

FIG. 16 is a front view of the smart phone, illustrating a daily exercise plan provided to a patient, according to one embodiment;

FIG. 17 is a front view of the smart phone, illustrating an exercise instructional video for a patient, according to one embodiment;

FIG. 18 is a front view of the smart phone, illustrating an avatar of a patient doing exercises, according to one embodiment;

FIG. 19 is a front view of the smart phone, illustrating a patient list for use by a physical therapist, according to one embodiment;

FIG. 20 is a front view of the smart phone, illustrating a detailed dashboard for one patient, for use by a physical therapist, according to one embodiment; and

FIG. 21 is a front view of the smart phone, illustrating an editable conditioning plan for editing by a physical therapist, according to one embodiment.

### DETAILED DESCRIPTION

In one embodiment, the systems, devices and methods described below are used to track pre-operative and/or post-operative progress in a patient who has undergone total knee replacement surgery (“TKR”). This embodiment is but one example of the systems, devices and methods of this disclosure, and the embodiment is not intended to limit the scope of the disclosure. In alternative embodiments, for example, the systems, devices and methods may be used with patients undergoing any interventional procedure on any joint, such as but not limited to arthroscopic procedures and open surgical procedures on the knee, shoulder, elbow, wrist, hip and ankle joints. In yet other embodiments, similar systems, devices and methods may be used (or modified for use) for providing patient information and communication regarding interventions that are not joint related, such as vascular procedures, spine surgery, neurosurgery, heart surgery and many other suitable interventions. Therefore, the following description should be interpreted as exemplary only and should not be interpreted as limiting the scope of the invention as defined by the claims.

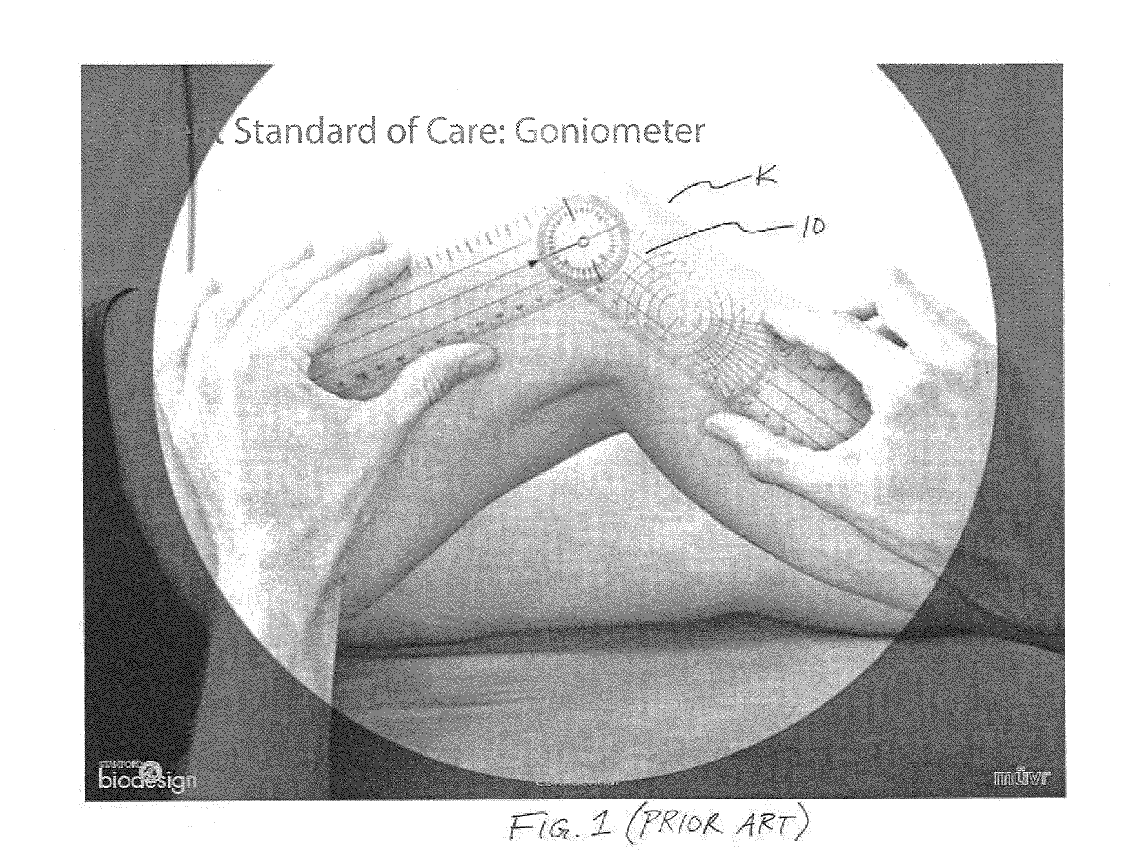

Referring now to FIG. 1, in the context of a TKR procedure, one of the commonly used prior art devices for measuring flexion and extension of the knee K is a simple goniometer 10. The goniometer is a very simple device that works like a protractor to measure an angle of the knee in flexion and extension. In other words, it helps measure how far a person can flex and extend his/her knee. Although a goniometer is simple and cheap, it is also very inaccurate and user dependent. The goniometer and a stopwatch to measure the timing of patient movements are often the only measurement tools at the disposal of a physical therapist for measuring patient progress after a TKR procedure or other knee surgery.

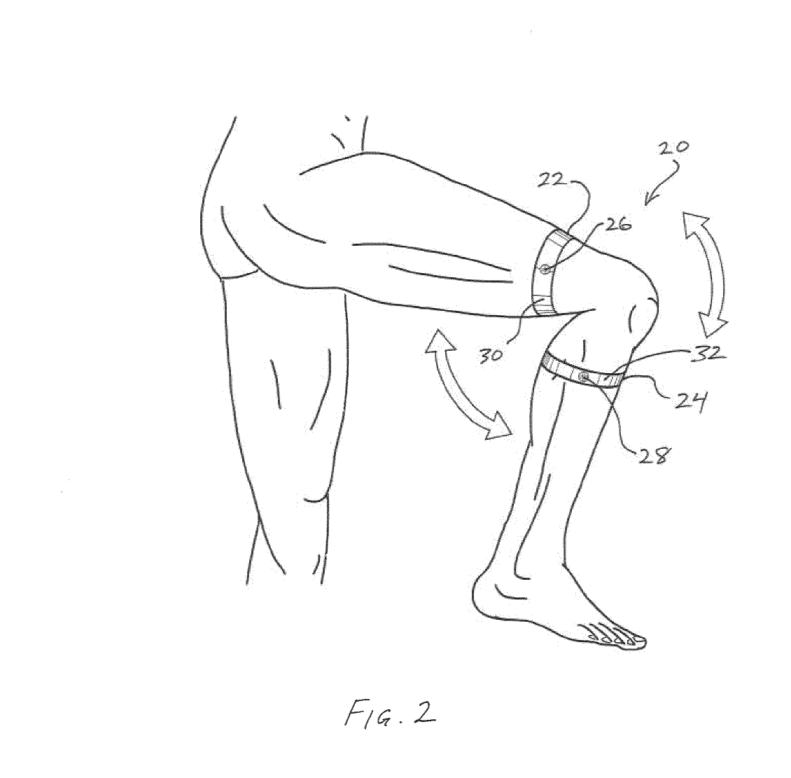

With reference now to FIG. 2, as mentioned above, in some embodiments, a joint sensor system 20 may be part of a larger system for providing patient feedback data and communicating with the patient. In this embodiment, monitoring system 20 includes two joint sensor devices—a first joint sensor device 22 configured for attachment to the patient's lower limb above the patient's knee joint K (over the femur) and a second joint sensor device 24 configured for attachment below the knee joint K (over the tibia/fibula). Each sensor device 22, 24 generally includes a sensor 26, 28 and an attachment mechanism, such as a strap 30, 32. Sensors 26, 28 may be any suitable motion sensor, such as but not limited to accelerometers, gyroscopes, magnometers, capacitive sensors, resistive sensors, optical sensors or any combination thereof. Each sensor 26, 28 is attached to the patient via strap 30, 32. In this embodiment, each sensor 26, 28 is also coupled with a microcontroller and a power source (not shown), which are also attached to the patient via strap 30, 32. In some embodiments, for example, sensors 26, 28, microcontrollers and power source may be housed in a small compartment on each strap 30, 32. The straps 30, 32 may be fastened by velcro, zipper, elastic, adhesive, compression fit or the like. Optionally, electrodes may also be attached to the patient via straps 30, 32, for example to measure electromyography signals from the patient's muscles.

The microcontroller may include a commercially available communication chip, with serial communications output through micro-USB, low energy Bluetooth, 802.11b/n Wi-Fi, infrared, radiowaves, microwaves, ultrasound, subsonic, audible frequency or other low-power communication signal, to export data to a personal computer, tablet device, mobile phone, Wi-fi repeater or other portable computing platform. Alternatively, the information may be transmitted over Wi-Fi or cellular signal to a cloud-based storage medium for processing in the cloud. This transmission may be directly from the sensor-microcontroller-communications combination chip to a mobile phone, tablet or computer, or through a Wi-Fi base station that then transmits the signal over Wi-Fi or cellular to the cloud-based storage system that can be accessed by a mobile phone, tablet or computer. All storage may be encrypted for security. The microcontroller, sensors 26, 28, and communication bridge may be powered by a lithium-ion battery or capacitive power supply. In some embodiments, the power supply may be rechargeable, such as by an inductive, wireless power charger or plug-in. The microcontroller may contain an internal clock for tracking the time and rate of sensor measurements.

In alternative embodiments, straps 30, 32 may be replaced with any suitable attachment mechanism for attaching sensors 26, 28 (and microcontrollers, power supply and/or other components of patient monitoring device 20) to the patient. In one alternative embodiment, for example, sensors 26, 28, microcontroller, and power supply may be integrated into a wound dressing made of silicone, cotton, hydrogel, and/or plastic. Alternatively the electronics may be affixed to the body with differently configured straps, adhesive or a fabric sleeve or article of clothing. Sensors 26, 28 may communicate with the microcontroller, power supply and communications bridge through conductors within or around the wound dressing or may be an imbedded system along with the microcontroller, power supply and communications bridge. In yet another alternative embodiment, sensors 26, 28, microcontroller and power supply may be integrated into a removable brace with adjustable straps, so that sensor position can be securely fixed over the femur and tibia. Ideally, the electronics of sensor devices 22, 24 may be waterproof and may be easily detached from, and reattached to, straps 30, 32 or other attachment mechanisms.

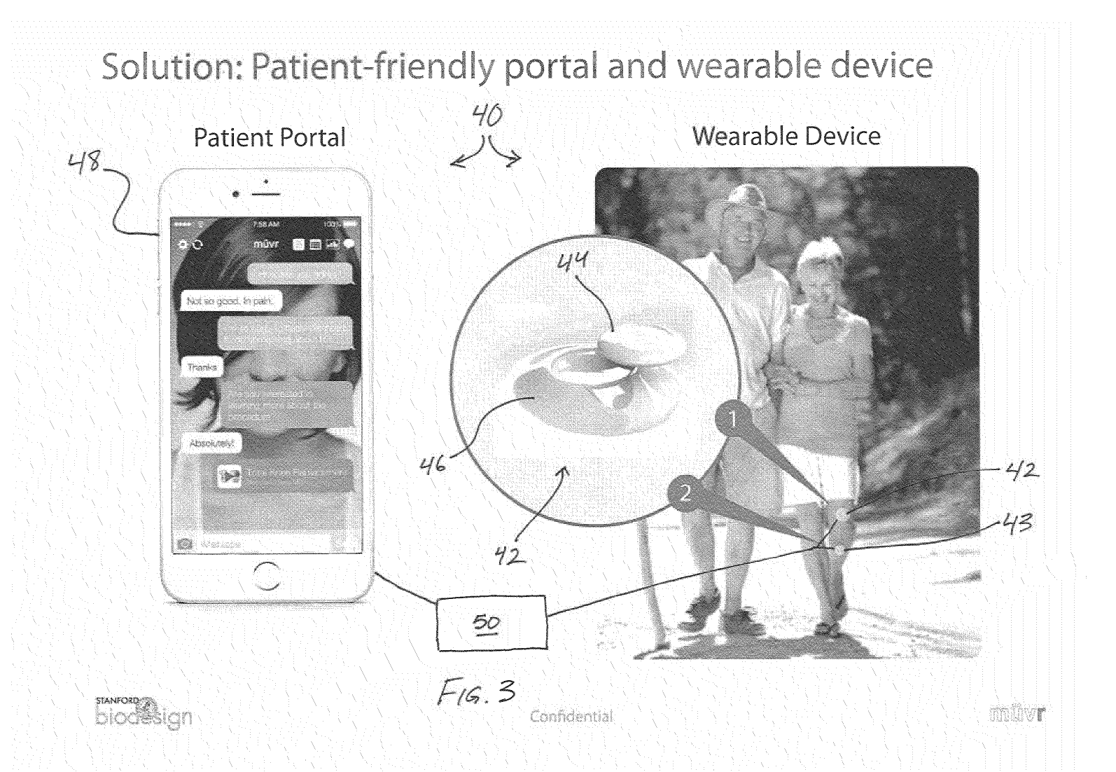

Referring to FIG. 3, one embodiment of a patient motion tracking and feedback system 40 may include multiple sensor devices 42, 43, a patient information portal 48, and a processor 50 coupled wirelessly with the sensors 42, 43 and the portal 48. In one embodiment, each sensor device 42, 43 may include a sensor electronics housing 44 (or “sensor pod”) and a base 46 (or “adhesive docking patch”). In some embodiments, most or all of the sensor electronics are disposed within the housing 44, and the base 46 is worn on the patient's skin, attached via an adhesive backing.

In various alternative embodiments, the housing 44 may range in size from approximately 1 cm to approximately 5 cm in diameter and may be between about 5 mm thick and about 3 cm thick. The housing 44 may contain features to aid in handling and grip, such as raised ridges and an indentation roughly the size of a thumbnail. Magnets in the housing 44 and the base 46 may provide a strong yet detachable connection between the two. Alternatively, the electronics housing 44 may snap into the base 46 through a press fit, may be screwed in by mating threads on the housing 44 and the base 46, or may be attached to one another by any other suitable means. In some alternative embodiments, the housing 44 and base 46 may be permanently attached or may be configured as a one-piece unit. The base 46 includes an adhesive surface for attaching to the patient's skin. In a typical embodiment, at least two sensor devices 42, 43, one on each side of a given articulation (or joint), would be used to measure range of motion of the joint. For example, secure fixation of a first sensor 42 above the knee (over the femur) and a second sensor 43 below the knee (over the tibia) may be used to communicate relative position between the sensors 42, 43 to the patient portal 48 (personal computer, mobile device, etc.), by means of the microcontroller and communications bridge to calculate knee range of motion.

In one alternative embodiment (not shown), the sensors, power supply, microcontroller, and communications device are embedded in an adhesive dressing. This dressing may incorporate commercially available wicking materials and antimicrobial agents, such as silver, at the surface that will contact the patient's skin at or near the joint. The size of the dressing may be variable, for example in some embodiments between approximately 4-10 inches long and approximately 1-6 inches wide. The dressing may be made of a malleable material, such as silicone, to be flexible, waterproof and breathable, and to conform to the peri-articular area. For increased accuracy, the sensors 42, 43 may be positioned near or over bony prominences or other fixed positions around the proximal and distal limb segments.

In alternative embodiments, the sensor devices 42, 43 may take the form of a strip, or elements woven into fabric or fabric-like material. In various alternative embodiments, the sensor devices 42, 43 may be worn on or inside clothes, may be strapped onto the patient, may be integrated into (or attachable onto) a knee brace or a knee support device. In one embodiment, sensors 42, 43 may be replaced by smart phone sensors. In various alternative embodiments, sensors 42, 43 may be attached to a belt or attached to or placed into pockets, shoes, socks, ankle bracelets, or the like, or embedded onto or into the skin, placed sub-dermally or implanted within the patient.

The patient portal 48 may be any suitable device that a patient may use to receive and optionally also transmit information from/to the system 40. In some embodiments, for example, the patient portal 48 may be a smart phone, tablet, laptop computer and/or other similar device. The processor 50 may reside in any of a number of different locations and/or devices. In some embodiments, for example, the processor 50 may reside in the cloud, and the sensors 42, 43 may transmit directly to the cloud. In other embodiments, processor 50 may reside in a base station located in the patient's home, a physical therapy office, a doctor's office, a hospital or any other suitable location. The base station may receive data from the sensors 42, 43 and may relay the data to a processor located in the cloud or elsewhere, either with or without pre-processing the data. In alternative embodiments, sensor data may be stored within or adjacent to the sensors 42, 43, or sensor data may be transferred to another sensor, to a smart device, such as a smart phone, or to a local or distant receiving station. The data may be processed within a system adjacent to the sensor system. The data may be processed in another sensor system, in a smart device or in a local or distant station. Any other suitable location and configuration for processor 50 is possible within the scope of the contemplated system 40.

Some embodiments of the system 40 may include a positioning device (not shown), to provide consistent and reliable positioning of tracking components, including the sensors 42, 43, on the patient's body. In one embodiment, a member of the care team or the user will place the tracking device(s) on the patient's body in or near the area of the wound and/or wound dressing. The placement device may be used to direct the placement of the tracking sensors 42, 43 to optimize functional accuracy, comfort and patient safety. The placement device may be a digitally guided system. For example a digital device may be used to guide the placement of the tracking device(s) with visual, audio or tactile cues. In an alternative embodiment, the positioning device may be a non-digital device. For example, the positioning device may look and function like a traditional knee brace. The patient (or healthcare profession) places the brace-like positioning device over the patient's joint, and the user may either attach tracking components using the placement device as a positional reference point, or the positioning device may have the tracking components pre-attached. In these embodiments, the patient simply removes the positioning device, and the tracking components remain in the desired location on the patient's body, for example with adhesive backing. Once the correct position of the two sensor devices on either side of a joint has been determined, the positions may be marked manually with an indelible marker or temporary tattoo. Alternatively, there may be indelible ink built into the initial calibration affixation adhesive devices to mark the spot for the replacement of the sensors through the course of care.

In some embodiments, the sensor devices 42, 43 may include a button to activate power and initiate communication with a personal computer, mobile device, tablet or Wi-Fi base station for transmission to the cloud or may be turned off and on remotely by connection with a mobile device or computer. Alterative power-on or activation mechanisms may also be used, such as motion via accelerometer or body warmth detection via thermocouple.

In various embodiments, any of a number of suitable users may be responsible for placing the tracking components, including the patient, the physical therapist, or other care providers. Ideally, the same design for the tracking device may be used by the different care providers. However, if appropriate, the placement device may be designed to accommodate the usability and constraints for these other care providers as well. For example, in the post-operative setting after the wound dressing has been removed, the user will have direct access to the skin near the wound area, which may be sensitive and unprotected. These sensitivities would be taken into consideration regarding the best way to place tracking components on the patient's body during this period. For example, the placement device may be designed to minimize contact with and pressure on the wound and the area near the wound.

A software algorithm on the processor 50, located on the personal computer, mobile device, cloud or the like, will process the data obtained from the sensors placed proximal and distal to each joint in question. The program can be calibrated using a manual or digital goniometer for reference. After calibration, the sensors 42, 43 may be configured to provide continuous or intermittent measurements of joint range of motion to the software platform. The software may then tabulate the results and report them to the provider per specifications set by the provider during setup of the software. Alternatively, the sensors 42, 43 may be calibrated by having the software guide the patient through several pre-set exercises. For example, the patient may be asked to walk up stairs then down and then sit in a chair and straighten the knee as much as possible then bend as much as possible. By doing these activities after indicating to the software that they are doing them, the software algorithm may calibrate the sensors 42, 43.

An alternative embodiment of a system for monitoring a patient and providing patient information (not shown) may include multiple, small, two-dimensional markers, such as circles, or three-dimensional markers, such as spheres, that are affixed to the patient around the joint with a fixed relation to each other. These markers may range in size from about 5 mm to about 20 mm in diameter, in some embodiments. Various embodiments may include anywhere from two to fifty markers for positioning around a joint. Using a single video camera from a mobile device or an array of up to 24 video cameras, video may be captured of the joint articulating through anticipated range of motion. The camera or camera array may be affixed to the proximal or distal limb segment to allow for measurement of distal or proximal limb segment motion, respectively. A software program may be calibrated with a manual or digital goniometer to enhance accuracy.

In some embodiments, calibration may be achieved by having the patient perform a set of tasks, such as sitting with the knees bent at 90 degrees, followed by standing with the knees in full extension. If the knees were not able to reach full extension or 90 degrees, this would have to be accounted for using other activities or a goniometer for calibration verification.

After calibration, the software may then be set to periodically download the sensor data from the microcontroller memory. The sampling rate of the sensors 42, 43 for the microcontroller may be manually adjusted and may determine when the memory becomes full and/or the frequency of downloads from the microcontroller memory to the personal computer or mobile device. For example, in one embodiment, the default sampling rate may start at 100 Hz and may be adjusted depending upon the application. The software on the personal computer or mobile device may be used to initiate and terminate sensing and recording as well as turning on and off individual sensors on the chip as they are needed. For example, if the gyroscope is not needed for a given task, it may be turned off to save power.

Once the sensor data has been exported to the personal computer or mobile device, the data would be stored and processed on the device or transferred to a cloud-based storage database platform for processing. In an alternative embodiment, the sensor data may be processed by a processor built onto the embedded chip, before data is transferred to another device or the cloud. Either way, the data may contain time stamps to allow the software to correlate the sensor measurements with activities either manually entered in the software by the user or activities prompted by the software. For example, the software may instruct the user to start a physical activity, such as ascending and descending stairs. Once the patient begins the activity, the sensor data would be recorded and linked to the specific activity. The data may then be queried for data such as maximum and minimum range of motion, including terminal extension and flexion, rate of joint motion in flexion and extension, activity type, time out of bed, number of steps taken, the time required by the patient to complete an activity and/or the like. In this way, the system 40 may be trained to identify any exercise and record relative metrics, such as maximum and minimum range of motion during the exercise, rates of knee flexion and extension, rates of motion of the leg and thigh segments, and time to complete the total exercise. The software on the computer, mobile device or cloud based storage platform may calculate the position of the leg at all times (using kinematic formulae), may correlate the motion with a specific activity by matching patterns to pre-set control patterns, may calculate the relevant metrics for the activity for retrieval by the application, and/or may calculate forces in the leg and thigh using inverse dynamics with the addition of a force monitoring plate in the shoes, such as a piezoelectric or capacitive based pressure transducer with blue tooth low energy communication to the phone or a Wi-Fi hub. The force plate provides the ground reaction force for greater accuracy in determining the leg, thigh and joint forces, along with other user input data, such as patient weight.

The software integrated with the device may contain games that encourage the user to increase range of motion, number of steps taken, time out of bed, stairs ascended and descended, time to ascend and descend stairs, six minute walk time, and the like. The data from the sensors 42, 43 may also be used to calculate joint position and leg-thigh position in real time and then be used to drive the motion of a virtual leg-thigh on a user's mobile device or computer. This data may also be used to drive a metric within a game using predetermined goals. For example, for knee range of motion, stretching to zero degrees may be visualized as a character trying to reach a cookie jar where the closer the user got to zero degrees, the further the hand would go in the cookie jar to get more cookies. Bending past 130 may represent a character diving deep for abalone where the deeper the knee bend, the more abalone the character would get. Users or care providers may define custom goals and/or customize/create other games, depending on the needs of the care provider and patient. The results of the games may be compared with anonymous usage data from other users that fit similar profiles as defined by care providers or users.

In some embodiments, the software may synthesize the data to provide relevant content for user dashboards. The dashboards used by the different users may contain the same information, or the content and user interfaces may be customized to the needs of the individual users or user types. For example, the physician and physician team may have an interface that enables them to track patients on an individual or a group basis. Similarly, physical therapists may have an interface that enables them to tailor physical therapy regimens, and communicate with the patient. Patients may have an interface that includes reminders for key activities like exercise, taking medication, and upcoming appointments, or interfaces that enable them to connect to a social community of other similar patients or relevant providers.

In various embodiments, the software may provide a range of probable outcomes for an individual patient based on an algorithm leveraging aggregated outcomes data of previous patients. The software may segment patients into specific risk profiles based on preoperative subjective and objective data collected through the software application and hardware sensors. Published algorithms on how to determine if patients are ready for surgery may be used to provide information as to if a patient is ready for total knee surgery, for example during shared decisions.

In some embodiments, the software may be set up by a physical therapist, sales representative, physician or the user. The physical therapy or exercise prescription would be input into the software per a physician's orders or per any other desired exercise regimen from a personal trainer, from a library of exercises that the user may choose from given their goals and desires or the like. The program or prescription may include frequency and timing of specific exercises relevant to the user's condition and goals. The software may notify a user, via alert, notification, pull up or down menu, application launch or the like, that it is time to do an exercise as prescribed per their physical therapy protocol. If the patient does not want to do exercises at that time, the patient may snooze the alert for period of time that may be adjusted depending on user preference. If the patient accepted doing the exercise, they may then use the application on their mobile device, tablet or computer to help them do their exercises properly, complete them and measure pre-determined mile stones such as range of motion goals, timed up and go, six minute walk time, time to ascend and descend stairs, total time out of bed or the like. These metrics may be pulled from the literature, for example. Videos may be available for each exercise, so that the user can choose to view a video to remind them how to do each exercise. The patient may also choose to enter a gaming mode that would allow their exercise to correlate to a game that would help them reach their goals. An avatar may also be shown that shows their activity in real time on the screen of their mobile device, tablet or computer.

If a patient is doing exercises incorrectly, as determined by the software algorithm, the patient may get an alert on the screen of a mobile device, computer, or any small display to direct them to correct the exercise. There may also be an array of colored LED's on the device itself to indicate if the exercise is being done wrong by displaying a red light, moderately good technique with a yellow light, and good technique with a green light. There may also be a vibration emitted from the device, if the patient is not doing the exercise properly. There may be several piezoelectric or similar vibration elements positioned on each side of a limb to help redirect the patient's exercises.

If the patient keeps snoozing their exercises, does not do them correctly, does not run the software or does not wear the device, the software may detect that the patient was not in compliance with the exercises that had been programmed into the software at initiation of the program. This may trigger an alert to the patient, for example via a phone call. There may be an alert to a pre-designated set of patient sponsors, such as family members, friends or coaches, who may then contact the patient to check in and see if there was a problem and to help get the patient back on track or escalate the situation to contact a healthcare provider. Alternatively, a practice manager, physical therapist, nurse practitioner, physician assistant or other designated allied health professional may be designated to be notified by text, email, call or alert on their mobile device if a patient was deviating from their care plan. Finally, the software may be programed to notify a physician in situations where that would be deemed necessary. These people may have a version of the software application on their device or computer that was set up for them to help monitor the user. When one of these persons is notified of the lack of compliance from a user, there may be a number of options for the sponsor individual to reach out to the user. A phone call, text message or videoconference may be initiated right from the application. The sponsor may also have the option to escalate the notification to an operator from the hospital, from our company or to the physician's office staff, patient navigator or other allied health professional should the sponsor feel that this may be necessary or if they are uncomfortable in any way with the way the patient is doing. The patient may also have the ability to reach out to the sponsor, physician staff, operator, physician or other appropriate party via text message including picture texting, email, videoconference or voice call directly from the mobile application or from a computer.

In addition to notifications about when to do exercises, the application may also prompt the patient with surveys at pre-set intervals set by the physical therapist and/or physician, per their specifications. For a total knee replacement patient, these surveys might include pain scores, such as the visual analogue scale, condition specific surveys, such as the WOMAC and KOOS, and general function scores, such as the SF-12 and SF-36. There may also be patient satisfaction scores given at specific times and regarding specific areas, such as satisfaction with pre-operative care, with care in the hospital, with care at home, with care in the outpatient setting and relative to various providers, including the surgeon, staff, inpatient and outpatient staff, and the like. This data may all be reported on the patient, therapist, physician and sponsor dashboards, and the specific data displayed in each dashboard may be customized for each practice during the setup period.

The system 40 will generally include the patient portal 48 with a main menu. This can be accessed from the Internet or via a mobile application. The menu may be customized for the condition for which the user is using the application. This customization may be completed by the implementation team, based on input from the patient and any other influencers, such as friends, coaches, physicians, physical therapists, nurses and/or other allied health professionals helping with the patient's condition. For example, in the case of a patient preparing for total knee replacement, they will be introduced to the mobile platform and web interface at a preoperative visit. This may be before the patient has decided to have knee replacement surgery, so that the application may help in shared decision making with their surgeon, or it may be given at a visit at a pre-designated time point before surgery in accordance with the physician and physical therapists pre-operative physical therapy protocol.

For example, during the initial setup of the application for a patient that is contemplating total knee replacement with a specific surgeon, the app may be set up with educational materials regarding the procedure, including tutorials, videos and quizzes to help the patient learn about the procedure, the anesthesia, the inpatient stay, outpatient process, recovery, physical therapy and/or any other educational materials the providers may want to incorporate. The main menu of the mobile app and web apps may include an education tab that the patient click to learn more. Sub-menus would direct the patient to the area of interest, such as a video of the procedure or a description of what to expect from anesthesia. During the initial setup, the patient may also setup their sponsors, such as a spouse, family member and/or others who will be willing to help them stick to their physical therapy exercises both pre- and post-op. During setup, the application may also be configured for the settings for a given orthopaedic practice in the case of total knee arthroplasty. This may include what educational materials they want specifically, what pre and post-op physical therapy protocols they would like to prescribe, how they would like to setup the chain of communication in the app, the frequency and timing of notifications, the snooze time, the timing of escalation to notify sponsors and providers, and the like. This information may all be set up during the initial setup, but may be modified later.

The main menu of the patient portal 48 may also include a button and submenu for communication. As outlined above, the patient may be able to choose to whom and in what way they would communicate. For example, they may choose to send an email question to the physician's secretary requesting to change an appointment or a picture of their wound may be text messaged to the surgeon's physician assistant. The main menu may also include a button for exercises. The patient may opt to start doing their exercises early, before being notified by the application.

The physical therapists for pre-op, in-hospital, home health, skilled nursing facilities, rehab facilities and outpatient physical therapy may be explicitly designated during the initial setup or a link to a website or mobile site may be generated for each therapist during the process to direct them to the website, mobile site and/or app that would be directed to the physical therapist. For therapists that have never used the system, the user specific key may grant them access to that patient's data through a secure web-portal and app. The portal setup for the therapist may contain training modules to orient the therapist with the software, its capabilities and how they can monitor the patient's progress and compliance through their dashboard, adjust the patient's therapy protocols through the software, and communicate with the patient through the portal as needed. The therapist may also communicate with prior and future therapists as well as other providers on the healthcare team through the app. The therapist may also use their interface for scheduling appointments and changing appointments as needed, to be closer or farther apart. The app may also help the therapist with documentation and billing as it may be customized to auto-populate the therapists required documentation with patient data and may be filled in online and exported for printing. In the future the app may also integrate with various electronic medical records to allow uploading of therapist documentation to the patient's record.

This hardware and software combination can be used for a variety of applications and use cases. It can be used for any patient that requires physical therapy. It is especially useful for cases where there is a finite period of therapy. Example cases include total knee replacement, ACL reconstruction surgery, meniscus surgery, hip replacement surgery, rotator cuff surgery, shoulder replacement surgery, ankle replacement surgery, limb deformity surgery, scoliosis surgery, elective spine surgery and more. Further applications include the field of physical fitness and sports therapy, especially in cases where there is a specific event that therapy or behavior is centered around, for example training for a marathon or other specific race.

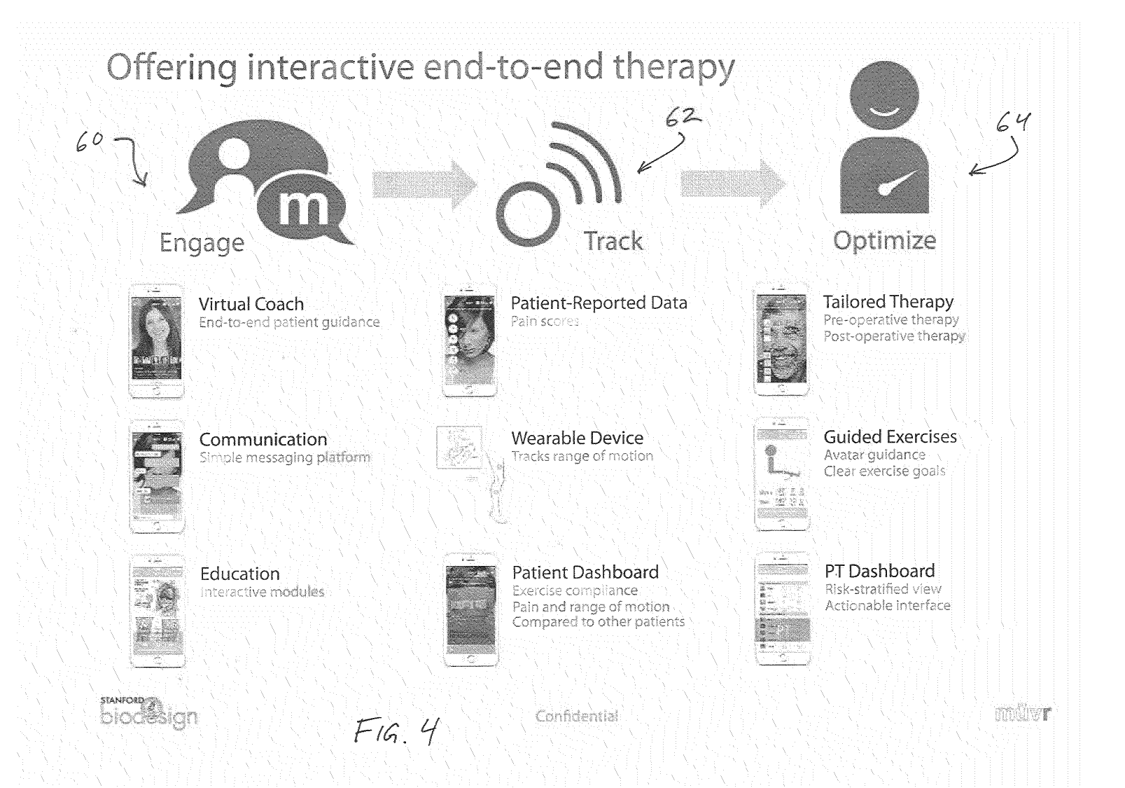

Referring now to FIG. 4, a method for communicating with a patient will be described. In general, the method may include three step—engaging with the patient 60, tracking one or more forms of data regarding the patient 62, and optimizing (or enhancing) the patient's therapy 64. Engaging the patient 60 may be achieved through communication via a smart phone or other device, as described above, and may involve communicating with a virtual coach, messaging between the patient and one or more care givers, and/or education. Tracking 62 may involve receiving patient-reported data, monitoring data from a patent wearable device, such as the sensor devices 42, 43 described above, and/or tracking a patient dashboard of information about the patient, such as exercise compliance. Optimizing or enhancing therapy 64 may involve providing therapy protocols specifically tailored for a patient, guiding exercises for the patient, and/or providing a physical therapy dashboard. These are only a few examples of the way in which the system 40 described above may be used. The following description, as well, is provided for exemplary purposes only and should not be interpreted as limiting the scope of this disclosure.

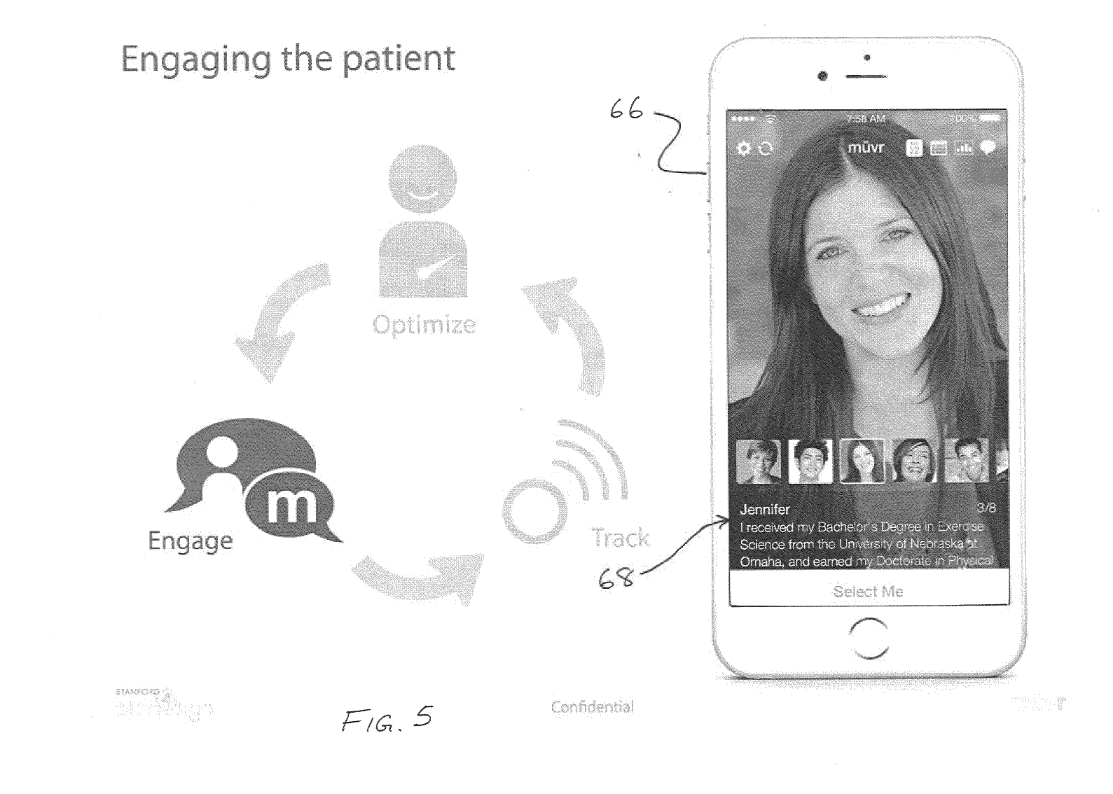

Referring now to FIG. 5, in one embodiment, a method for communicating with a patient may start by engaging with the patient via a smart phone 66 or any other suitable communication device. For example, one way to engage with the patient is to introduce one or more care providers (physician, nurse, physical therapist, etc.) to the patient. This introduction may include a short bio 68 of the person being introduced, for example, and may be provided before, during or after the patient has met with the surgeon, in deciding to undergo surgery. In some embodiments, the patient may be able to select a virtual coach to work with through the process. The coaches may be real people, such as physical therapists, patient navigators, physicians, care providers, or other individuals. Alternatively, the coaches may be automated systems, designed to respond appropriately to the user. The coaches may interact via video. The coach images may change on the app screen to reflect different expressions. For example, to indicate sympathy, the coach image shown may be a sad expression. Or to indicate excitement, the coach image used may be a happy or enthusiastic expression. This may be used with the communication component of the app, where the coach expression changes to reflect the content of the dialogue. This feature may also be used throughout the various patient interactions to reflect the nature of the interaction.

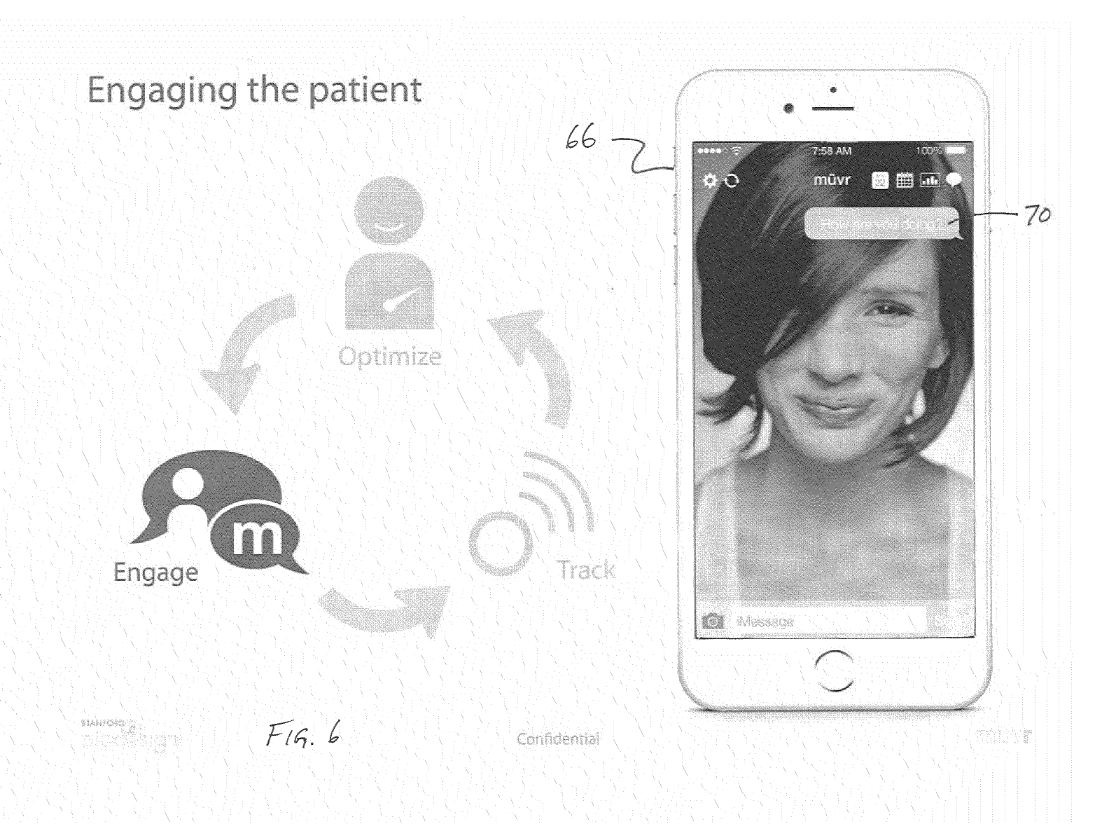

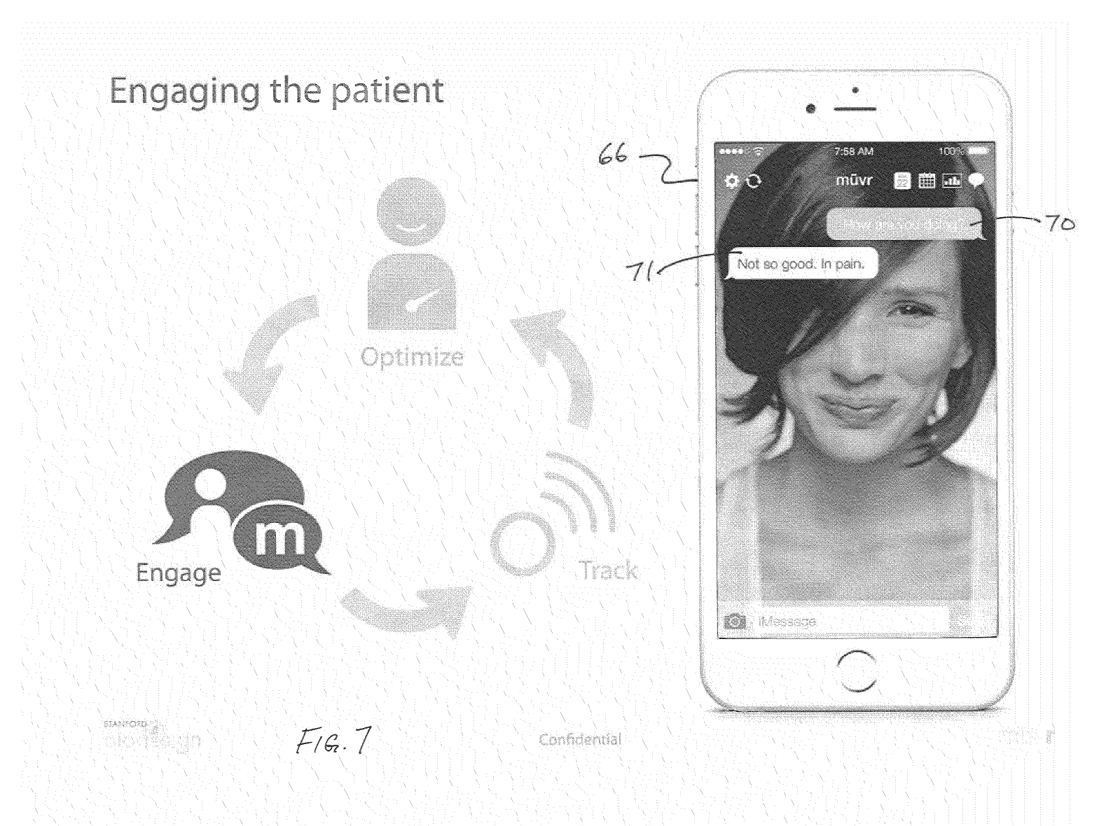

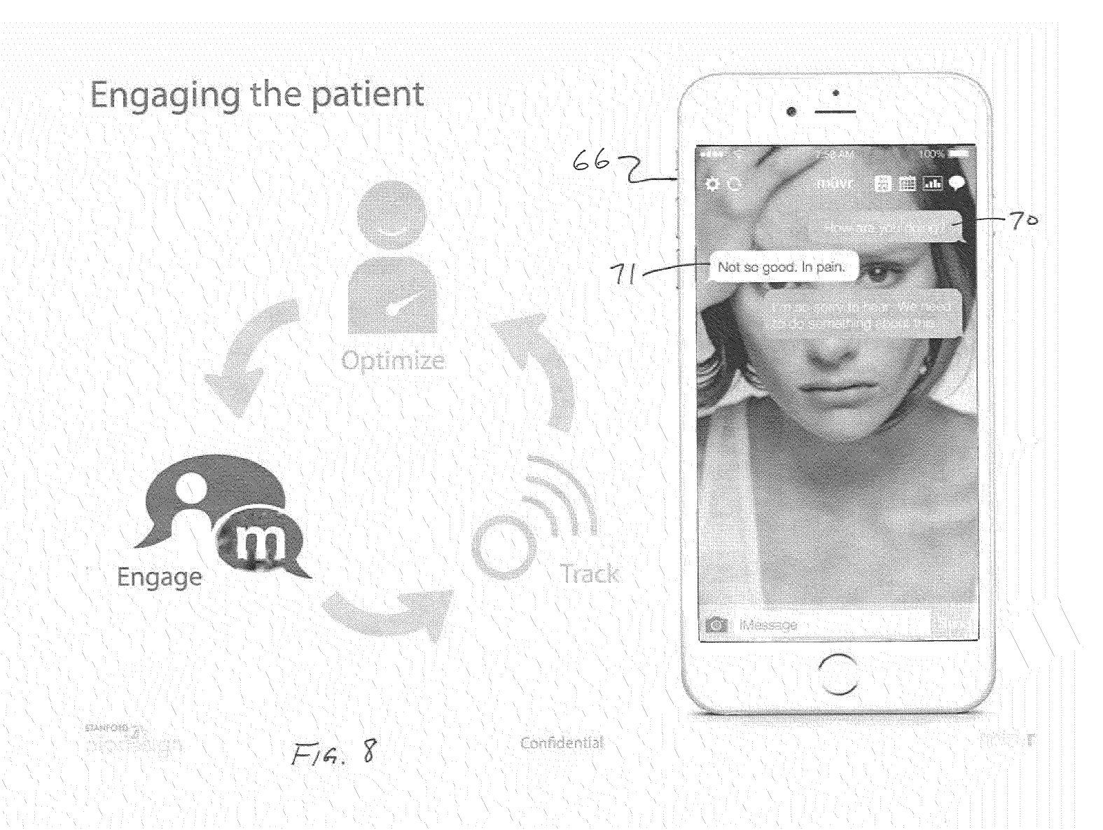

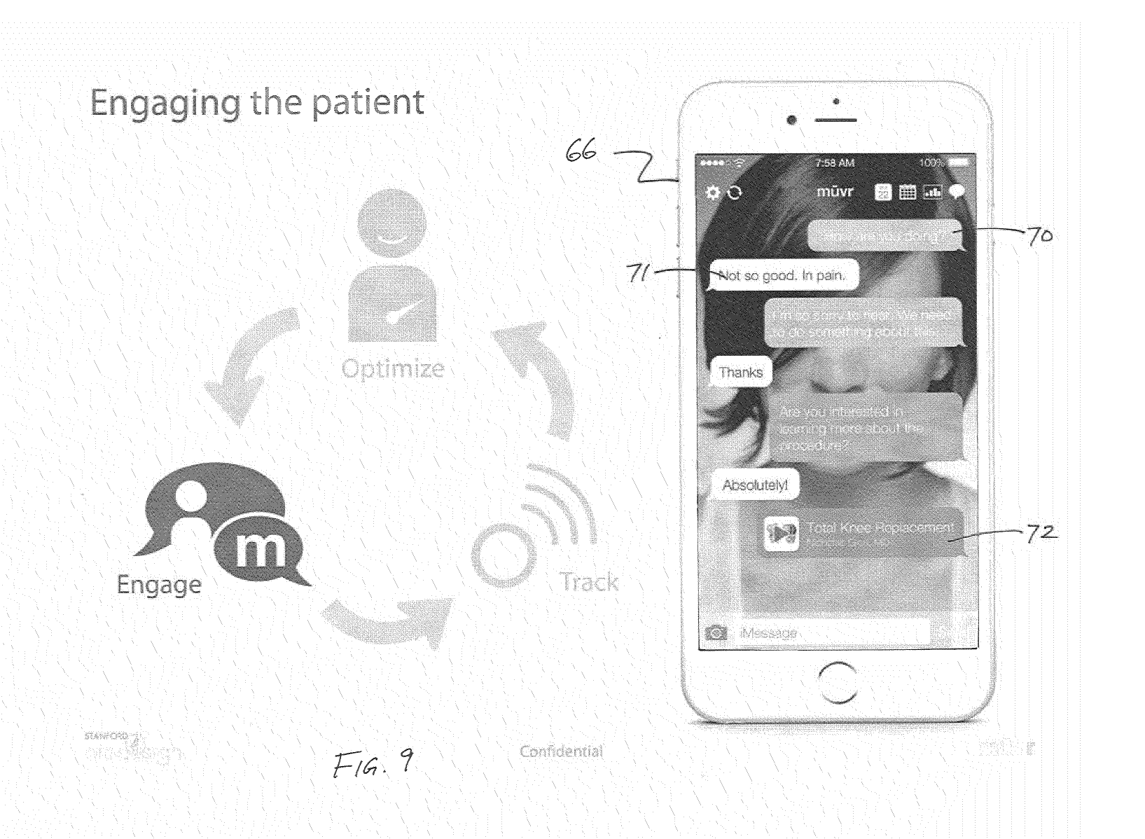

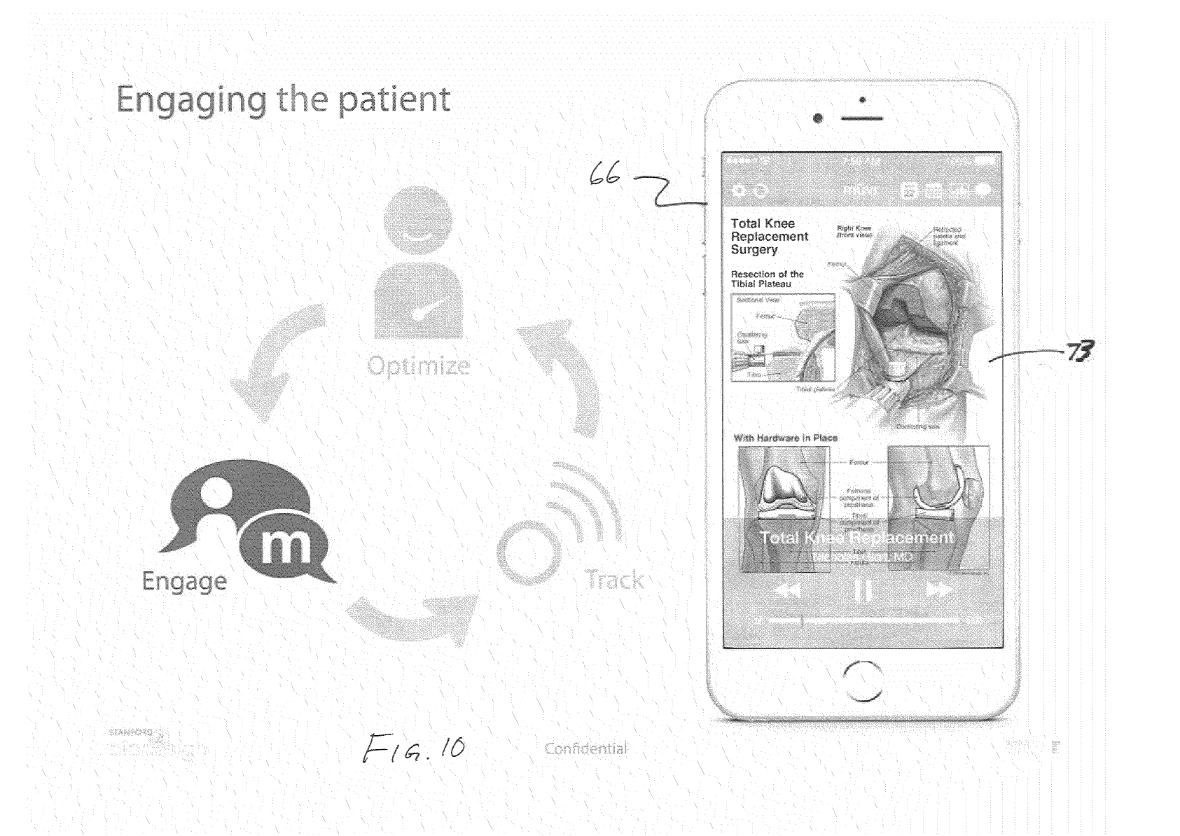

With reference now to FIGS. 6-10, in some embodiments, engaging the patient may include talking with the patient via text messaging and/or any other suitable media. The smart phone 66 may be used, for example, to send the patient an initial message 70, such as an inquiry about the patient's health. The patient may respond 71, and a conversation may ensue. At some point in the conversation, as in FIG. 9, the patient may be provided with a link to further information 72, such as educational material about a surgical procedure. FIG. 10 is a screen shot of an educational page 73 on total knee replacement surgery, as one example. The format of the educational material may be any suitable format, such as video, still images, text based and/or interactive digital. There may be testing, quizzing and/or other interactive features to test the patient engagement and understanding.

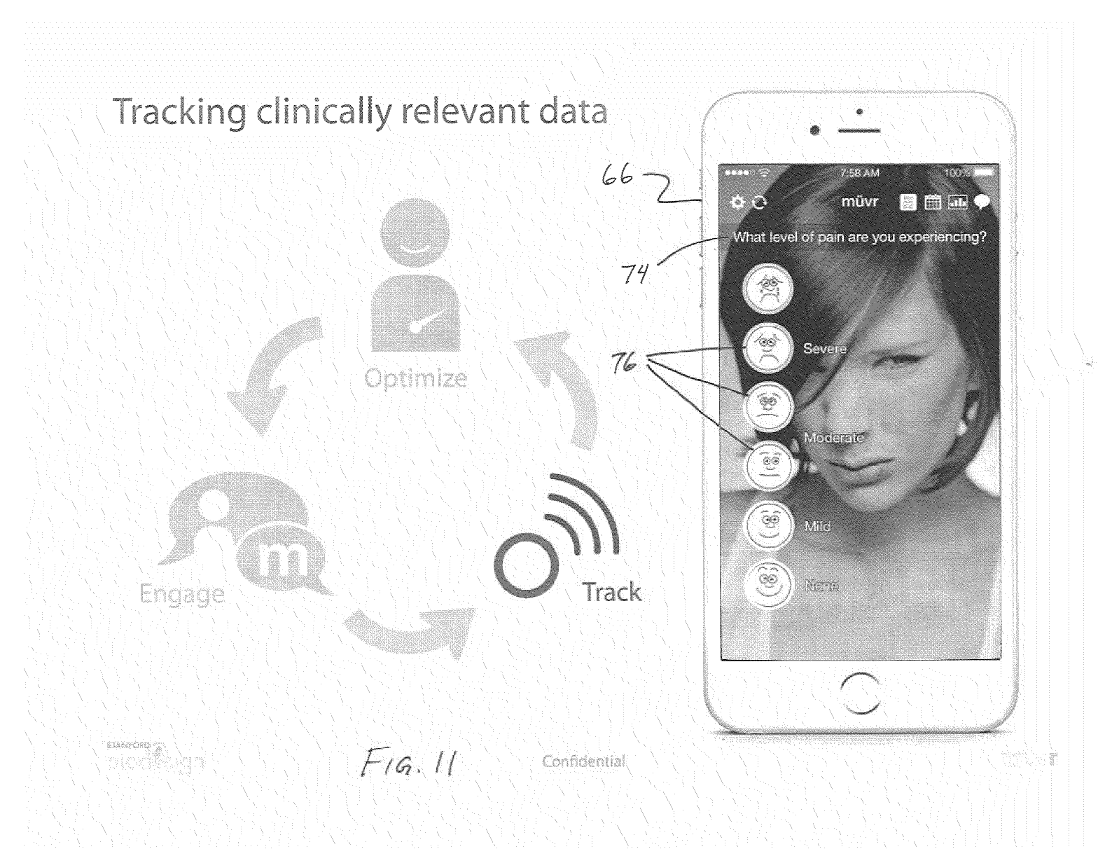

Referring now to FIGS. 11-13, the method may also involve tracking patient data 62, as mentioned above. One example of tracking, as illustrated in FIG. 11, may involve asking the patient a question about her current status 74 and providing buttons 76 or other input prompts to allow the patient to submit a response. The app may use this type of interaction or any other suitable means to track patient-reported data. This data may include pain scores, patient satisfaction scores, drug prescriptions and interactions, visual analog scores, disease specific WOMAC and KOOS, non-disease specific input, such as SF-12 and SF-36, and/or other patient-reported information.

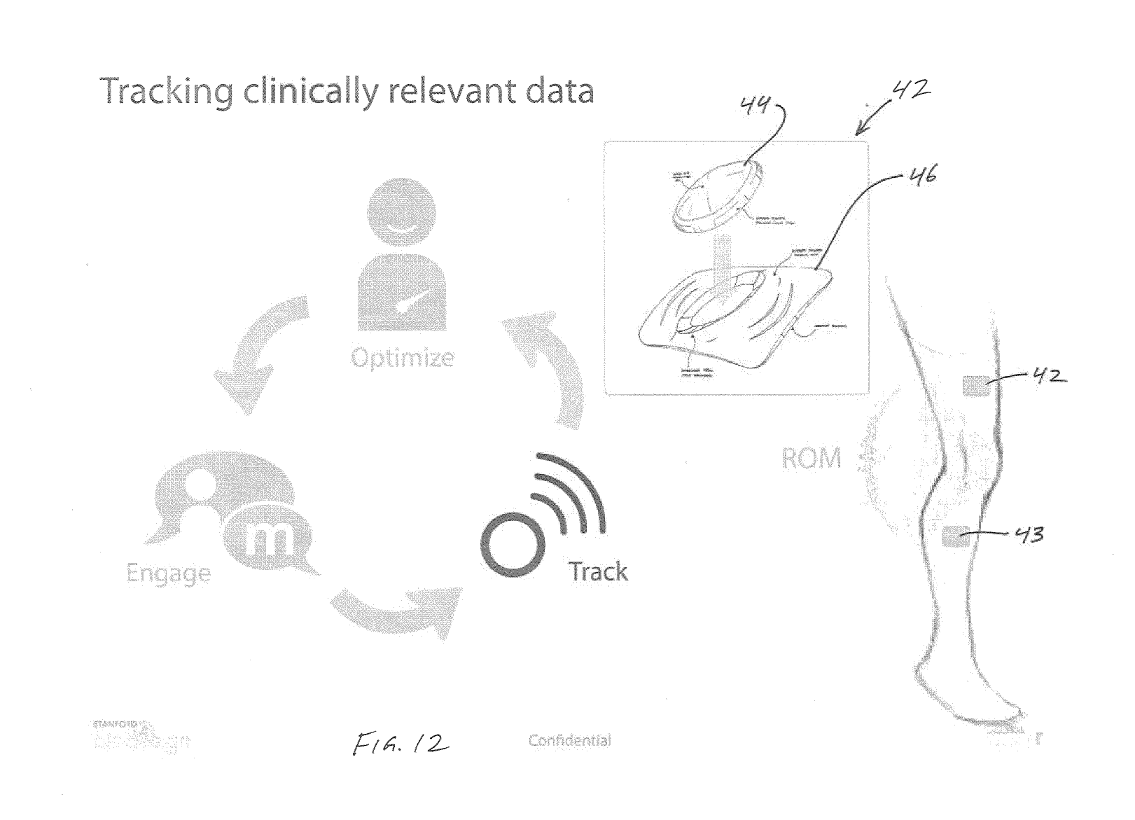

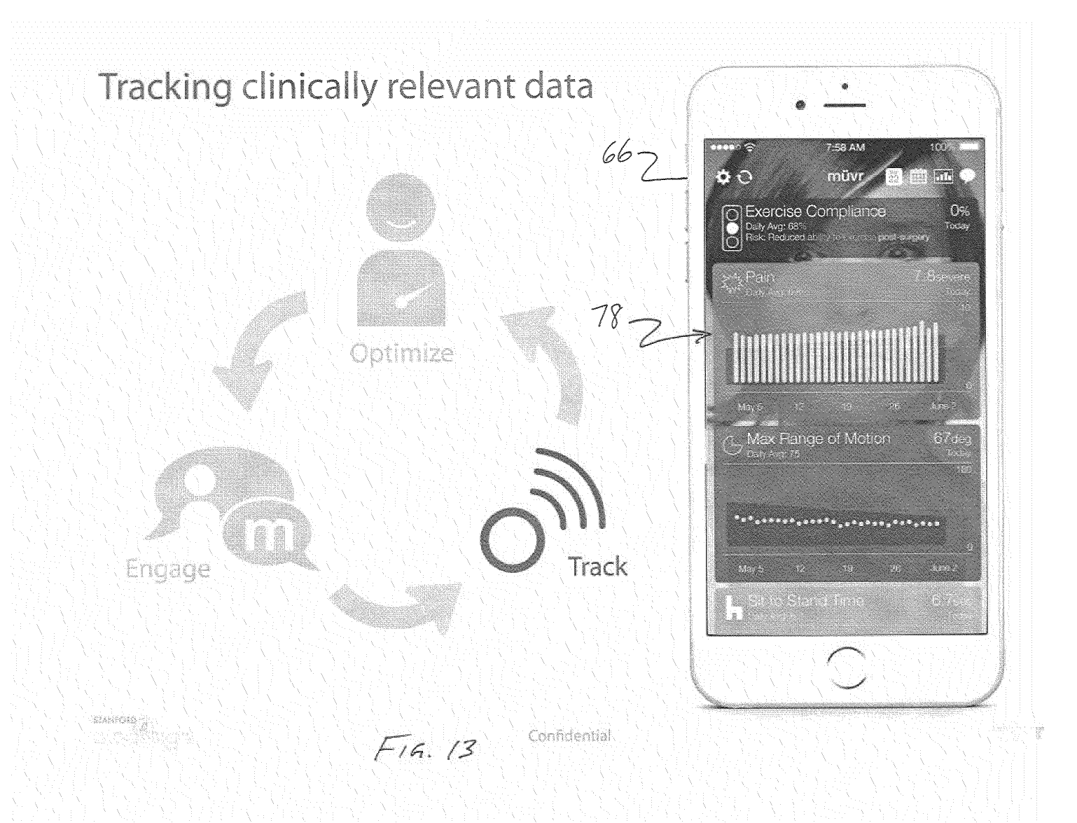

Referring to FIG. 12, tracking may also include tracking data from sensor devices 42, 43 placed on the patient, as described in detail above. Referring to FIG. 13, the data collected from the patient and from the sensor devices 42, 43 may be used in one or more dashboards 78. Displayed data may include exercise compliance, general activity, stairs climbed or descended, distance traveled, steps taken, pain over time, range of motion over time, and drug interactions. Other metrics that may be displayed include sit-to-stand time, time up and go, tug test, 6-minute walk, time to descend/ascend stairs, maximum extension/flexion, time out of bed and/or the like. This data may be used to inform or incentivize behavior.

Finally, in addition to engaging the patient 60 and tracking the patient 62, the method may also include providing information to the patient to help optimize or enhance the patient's therapy 64. The data collected from the patient and from the device(s) may be used to design tailored therapy plans for the patient. The base plan may be a validated protocol, and may come from an institution, such as Stanford or Harvard. The therapy plan may be adjusted to individual patients, depending on the patient-specific condition. For example, patients who are more sedentary and/or experience more pain may benefit from a decreased exercise regimen, and patients who are more young and active may benefit from an increased or maximized exercise regimen.

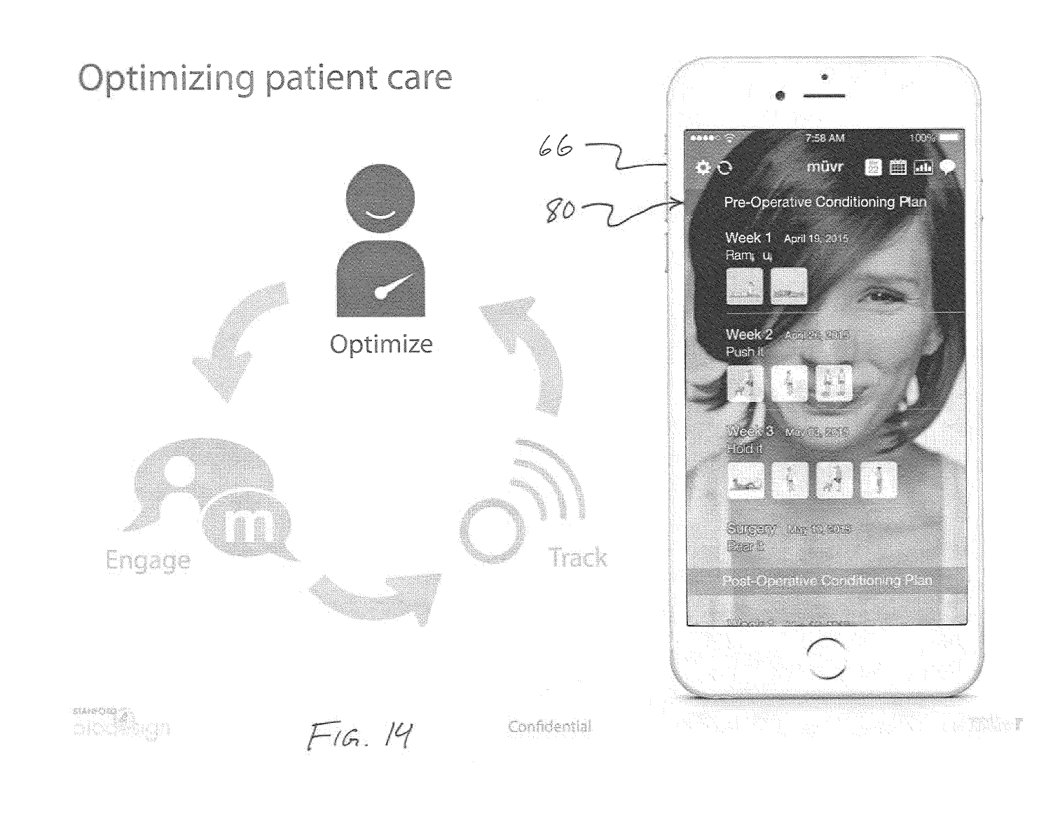

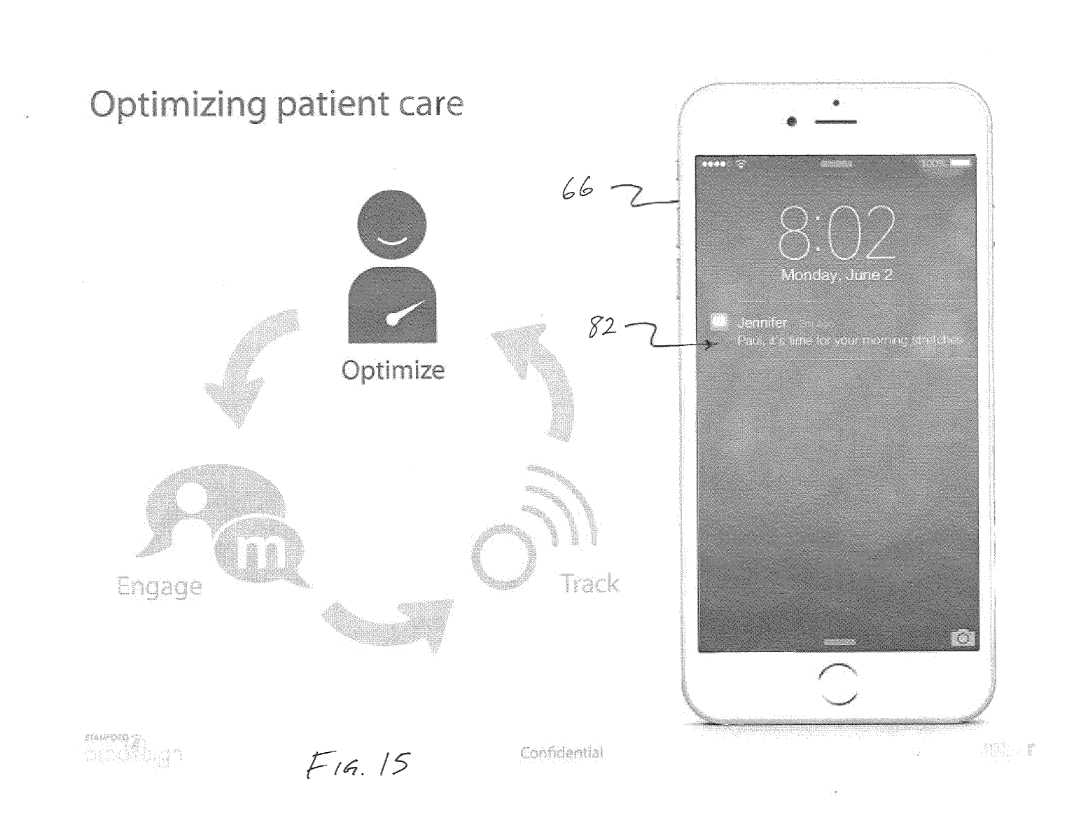

Referring to FIG. 14, in some embodiments, each patient may be provided with pre-operative and/or post-operative conditioning plans 80. Referring to FIG. 15, in some embodiments, patients may receive notifications 82 as part of the therapy enhancement portion of the method. Notifications 82, for example, may remind patients to do their exercises, remind patients of upcoming appointments and/or remind patients of other activities and tasks.

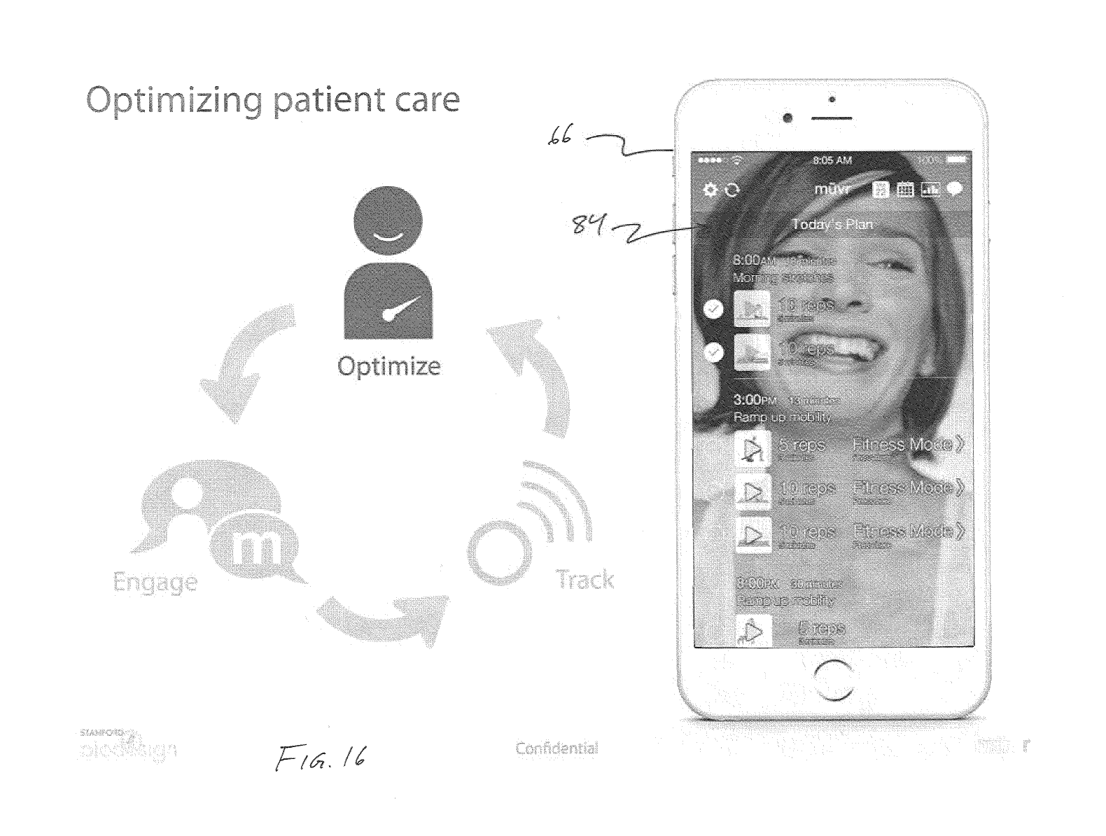

Referring to FIG. 16, in some embodiments, the patient may receive an individually-tailored daily plan 84. The plan 84 may display timing for therapy and exercises. The plan 84 may include instructions or links to instructions, such as videos for therapy. The plan 84 may include guidance for therapy, such as number of sets, reps or other therapy goals, such as range of motion goals. The plan 84 may include check boxes to indicate if an exercise or therapy has been completed. The boxes may be automatically checked based on sensor-derived data. The plan 84 may include links to features such as “fitness mode” or other support features to guide the patient through therapy.

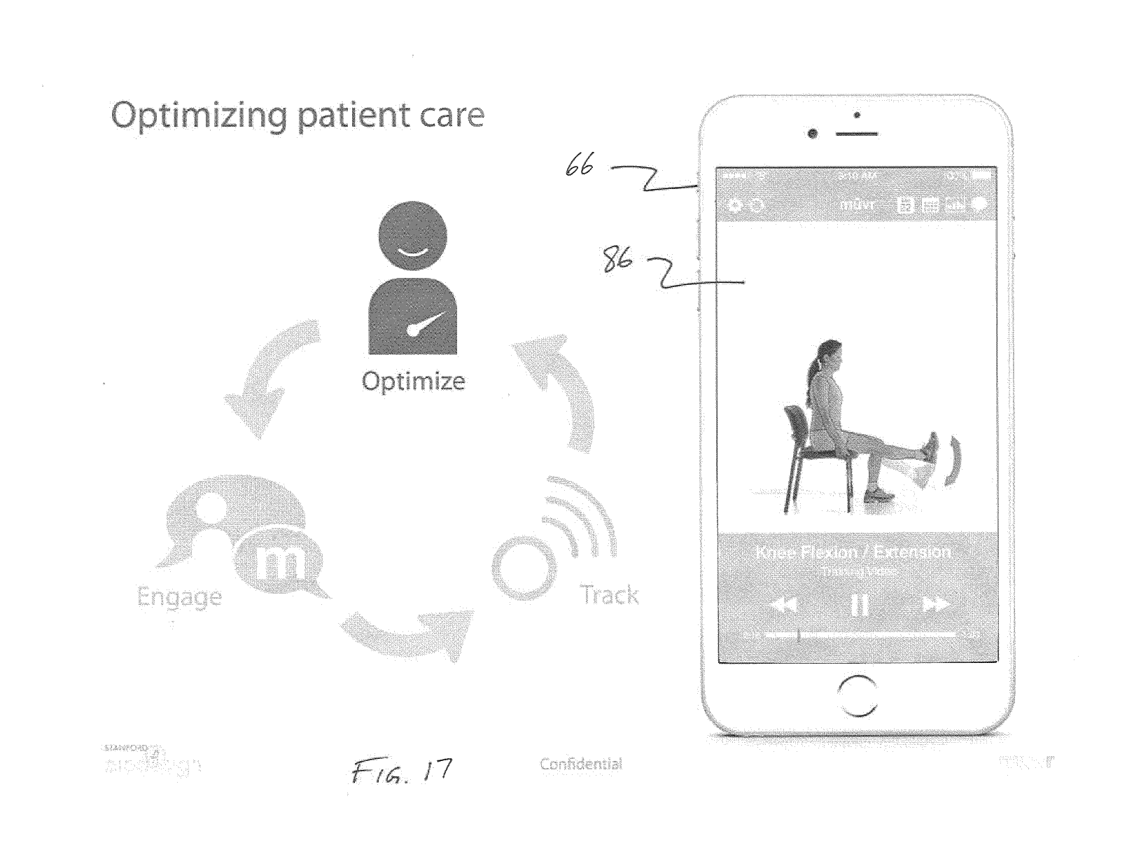

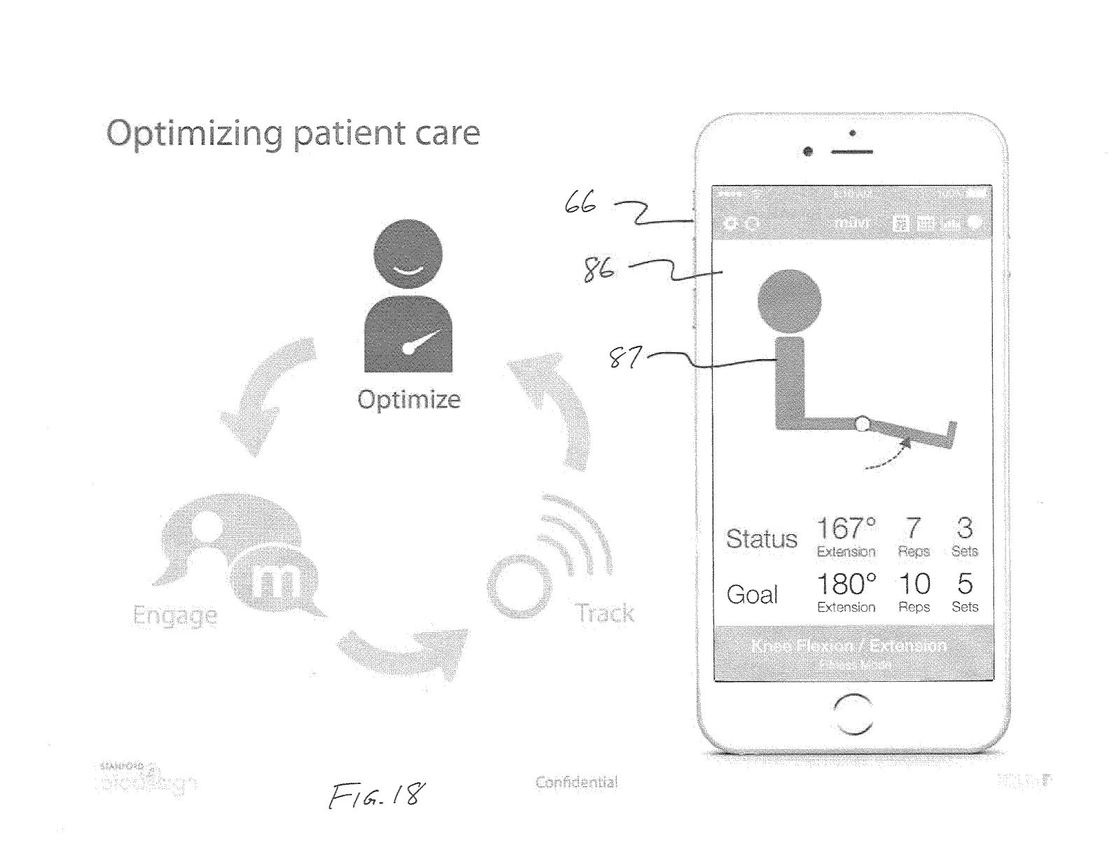

Referring to FIG. 17, in some embodiments, an app 86 may be provided, which includes video modules to guide the patient in therapy and/or exercises. This content may be presented in the form of text, images and/or interactive content. Referring to FIG. 18, in some embodiments, the app 86 may include exercise guidance features, such as an avatar 87. The avatar 87 may mimic patient movement and orientation, and it may do so in real-time or close to real-time. The avatar feature 87 may also include therapy goals and status.

In some embodiments, devices may be included to physically guide patient through therapy. These features may be integrated into the previously described sensor devices 42, 43. Guidance may be provided to the patient through vibrating components, light-based components, or digital components, such as audio, visual and/or tactile components on a smart device.

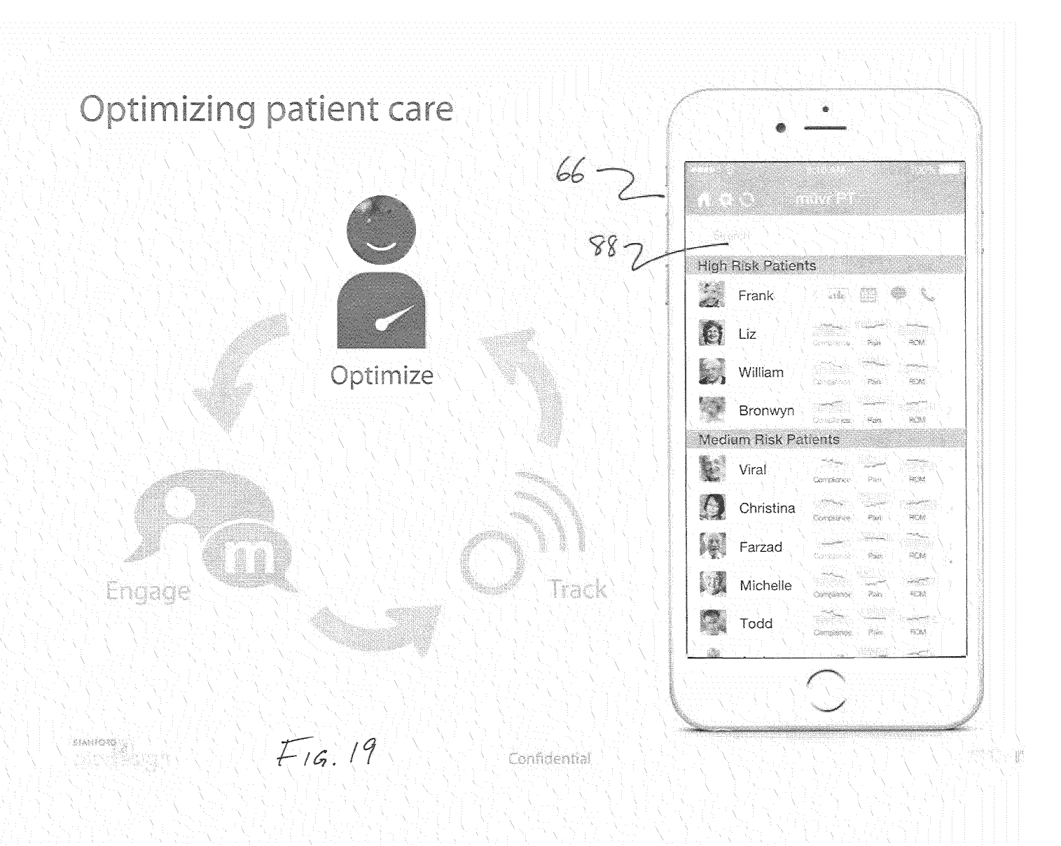

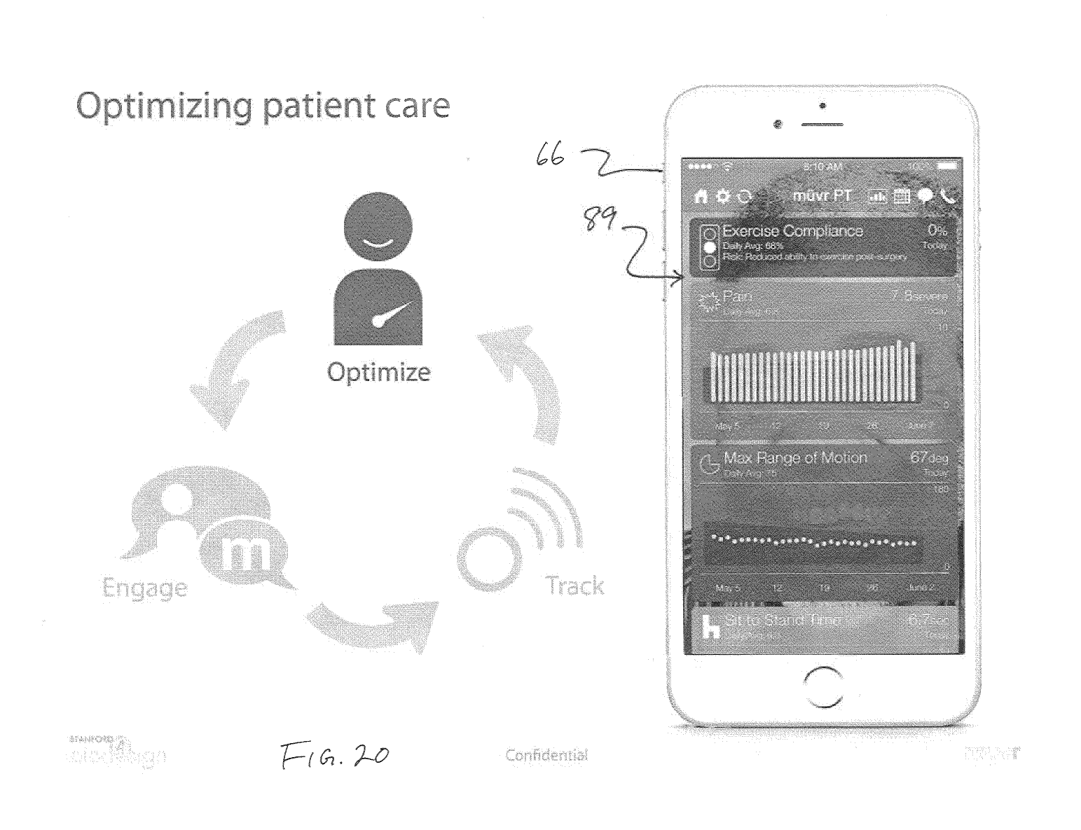

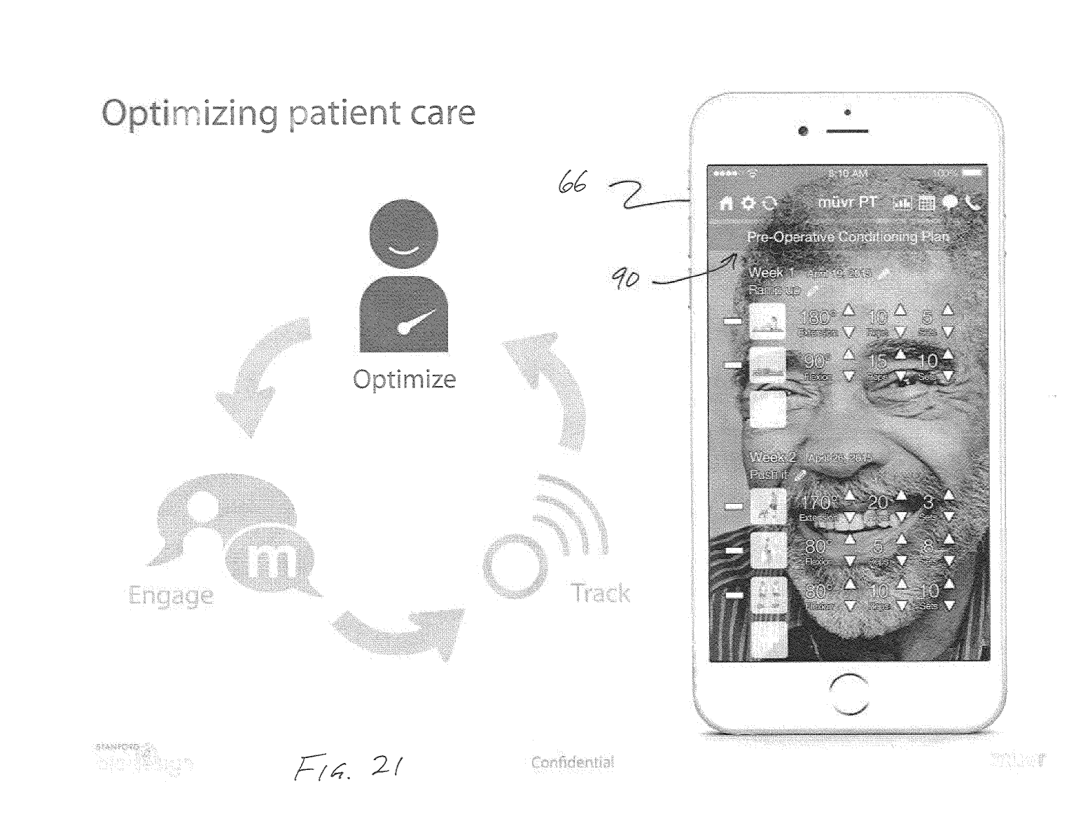

Referring now to FIG. 19, in some embodiments, the method may include providing an app 88 configured for use by physical therapists (PTs). The app 88 may include a dashboard of patients, stratified by risk. Information may include compliance, pain and range of motion. Referring to FIGS. 20 and 21, in some embodiments, PTs may be able to act on this information by accessing a more detailed dashboard set 89 (FIG. 20), by communicating directly with the patient via text or phone, or by editing the patient therapy regimen 90 (FIG. 21). This may include removing, adding or adjusting therapy.

It is intended that the scope of the present methods and apparatuses be defined by the following claims. However, this disclosure may be practiced otherwise than is specifically explained and illustrated, without departing from its spirit or scope. Various alternatives to the embodiments described herein may be employed in practicing the claims, without departing from the spirit and scope as defined in the following claims. The scope of the disclosure should be determined, not with reference to the above description, but instead with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art,unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. A system for providing information to a patient and receiving information from the patient before and/or after a medical or surgical procedure, the system comprising:
a joint motion sensor system, comprising:
a first sensor device configured to be coupled with the patient above a joint and including a first transmitter for transmitting sensed data from the first sensor device; and
a second sensor device configured to be coupled with the patient below the joint and including a second transmitter for transmitting sensed data from the second sensor device;
a processor configured to receive sensed data from the first and second sensor devices and process the sensed data to provide joint motion data; and
a third transmitter coupled with the processor for transmitting the joint motion data wirelessly to the patient.

2. A system as in claim 1, wherein the medical or surgical procedure comprises a total knee replacement surgery, and wherein the first and second sensor devices are configured to be coupled above and below a knee joint of the patient.

3. A system as in claim 1, wherein the joint motion data comprises an angle of flexion of the knee joint and an angle of extension of the knee joint.

4. A system as in claim 1, wherein the joint motion sensor system is configured to be coupled with the patient on either side of a joint selected from the group consisting of a knee, an ankle, a hip, a wrist, an elbow and a shoulder.

5. A system as in claim 1, wherein the first sensor device and the second sensor device each comprises:
a sensor electronics housing; and
a base for removably coupling with the housing, wherein the base comprises a housing attachment surface and an adhesive surface for attaching to the patient's skin.

6. A system as in claim 1, further comprising an app for providing the joint motion to the patient.

7. A method for providing information to a patient and receiving information from the patient before and/or after a medical or surgical procedure, the method comprising:
receiving patient information in a processor before the patient undergoes the medical or surgical procedure;
providing information to the patient via an electronic communication device before the patient undergoes the procedure, at least partially in response to the received patient information;
receiving sensed data from at least one sensor attached to the patient after the medical or surgical procedure;
processing the sensed data to generate the patient feedback data; and
transmitting the patient feedback data wirelessly to the electronic communication device for use by the patient.

8. A method as in claim 7, wherein receiving the patient information comprises receiving a message from the patient describing a state of a health condition of the patient.

9. A method as in claim 7, wherein providing information to the patient via the electronic communication device comprises providing information via a device selected from the group consisting of a smart phone, a tablet, a laptop computer and a desktop computer.

10. A method as in claim 7, wherein providing information to the patient via the electronic communication device comprises providing a pre-procedure physical therapy plan to the patient.

11. A method as in claim 7, further comprising providing a post-procedure physical therapy plan to the patient via the electronic communication device.

12. A method as in claim 11, further comprising adjusting the post-procedure physical therapy plan, based at least in part on the patient feedback data.

13. A method as in claim 7, wherein receiving the sensed data comprises receiving joint motion data from at least two joint motion sensor devices attached to the patient near one of the patient's joints.

14. A method as in claim 13, wherein the medical or surgical procedure comprises a total knee replacement surgery, and wherein the sensed data comprises joint motion data related to flexion and extension of one of the patient's knees.

15. A method as in claim 13, wherein receiving the sensed data comprises receiving joint motion data sensed while the patient is exercising, and wherein the patient feedback data comprises data describing whether the patient is performing one or more exercises correctly.

16. A method as in claim 7, further comprising transmitting the patient feedback data to at least one of a physician, a nurse or a physical therapist.

17. A method as in claim 7, further comprising receiving patient input in the processor after the medical or surgical procedure.

18. A method as in claim 7, wherein receiving the patient information and providing the information comprises receiving and sending text messages to and from the patient, respectively.

19. A method as in claim 7, further comprising providing patient guidance to the patient after the medical or surgical procedure, wherein the patient guidance is selected from the group consisting of a post-surgical physical therapy plan, a post-surgical exercise plan, instructional information on how to perform one or more exercises and an avatar illustrating how the patient is performing one or more exercises.

20. A method as in claim 7, further comprising providing an electronic dashboard to a physical therapist working with the patient to track progress of the patient in a post-surgical physical therapy plan.
